(19) United States
(12) Patent Application Publication
(10) Pub. No.: US 2009/0139528 A1
(43) Pub. Date: Jun. 4, 2009

(54) Tactile Apparatus and System for Oxygen Tube

(76) Inventor: Olga Yevich, Shelton, CT (US)

Correspondence Address: LAW OFFICE OF MICHAEL A. BLAKE, LLC, 95 HIGH STREET, SUITE 5, MILFORD, CT, 06460, US

(21) Appl. No.: 11/949,897

(22) Filed: Dec. 4, 2007

(51) Int. Cl. A61M 16/04

(52) U.S. Cl. 128/207.14

(57) ABSTRACT

A tactile system for an oxygen tube attachable to a tracheostomy mask, the system comprising: an oxygen tube; an oxygen tube cover removeably attached to the oxygen tube, the oxygen tube cover comprising: a first end; a second end located on an end opposite the first end; a width defined generally by the distance along the oxygen tube cover from the first end to the second end; a length; an inner surface; an outer surface located on a surface opposite the inner surface; a plurality of elastic bands attached to the oxygen tube cover generally parallel to the width; a first connecting means located on the inner surface and generally at the first end; a second connecting means located on the outer surface and generally at the second end; and where the oxygen tube cover is generally made of a soft material. A tactile apparatus for an oxygen tube attachable to a tracheostomy mask, the tactile apparatus comprising: an oxygen tube cover removeably attachable to an oxygen tube, the oxygen tube cover comprising: a first end; a second end located on an end opposite the first end; a width defined generally by the distance along the oxygen tube cover from the first end to the second end; a length; an inner surface; an outer surface located on a surface opposite the inner surface; a plurality of elastic bands attached to the oxygen tube cover generally parallel to the width; a first connecting means located on the inner surface and generally at the first end; a second connecting means located on the outer surface and generally at the second end; and where the oxygen tube cover is generally made of a soft material.

### TECHNICAL FIELD

This invention relates to a tactile apparatus and system for an oxygen tube used with a tracheostomy tube, and more particularly to a tactile apparatus and system for an oxygen tube that is soft and pleasing to the touch.

### BACKGROUND

Patients with respiratory obstructions that interfere with normal breathing are often treated with tracheostomy surgery to provide an opening or stoma through the neck and into the trachea whereby a tracheostomy tube can be inserted to create an alternative breathing passageway. The tracheostomy tube may be in fluid communication with an oxygen tube which supplies oxygen to the patient. The oxygen tube is typically located in reach of the patient. When the patient is very young, such as a baby or a toddler, the patient is often told to not touch or pull at the oxygen tube, for fear of interfering with the oxygen supply to the patient. In fact, young patients are so often told not to touch the oxygen tube, that the young patient learns to stop exploring the world around him or her, and may eventually lead to an introverted, inactive, disengaged child.

Thus there is a need for an invention that can help prevent a child from constantly being told to not touch his or her oxygen tube.

### SUMMARY

The disclosed invention relates to a tactile system for an oxygen tube attachable to a tracheostomy mask, the system comprising: an oxygen tube; an oxygen tube cover removeably attached to the oxygen tube, the oxygen tube cover comprising: a first end; a second end located on an end opposite the first end; a width defined generally by the distance along the oxygen tube cover from the first end to the second end; a length; an inner surface; an outer surface located on a surface opposite the inner surface; a plurality of elastic bands attached to the oxygen tube cover generally parallel to the width; a first connecting means located on the inner surface and generally at the first end; a second connecting means located on the outer surface and generally at the second end; and where the oxygen tube cover is generally made of a soft material.

The disclosed invention also relates to a tactile apparatus for an oxygen tube attachable to a tracheostomy mask, the tactile apparatus comprising: an oxygen tube cover removeably attachable to an oxygen tube, the oxygen tube cover comprising: a first end; a second end located on an end opposite the first end; a width defined generally by the distance along the oxygen tube cover from the first end to the second end; a length; an inner surface; an outer surface located on a surface opposite the inner surface; a plurality of elastic bands attached to the oxygen tube cover generally parallel to the width; a first connecting means located on the inner surface and generally at the first end; a second connecting means located on the outer surface and generally at the second end; and where the oxygen tube cover is generally made of a soft material.

### BRIEF DESCRIPTION OF THE DRAWINGS

The present disclosure will be better understood by those skilled in the pertinent art by referencing the accompanying drawings, where like elements are numbered alike in the several figures, in which:

FIG. 1 is a perspective view of a tracheostomy mask and oxygen tube;

FIG. 2 is a perspective view of the tracheostomy mask from FIG. 1 with an oxygen tube cover attached to the oxygen tube;

FIG. 3 is a perspective view of the oxygen tube cover in an open configuration; and

FIG. 4 is a perspective view of another embodiment of the disclosed oxygen tube cover.

### DETAILED DESCRIPTION

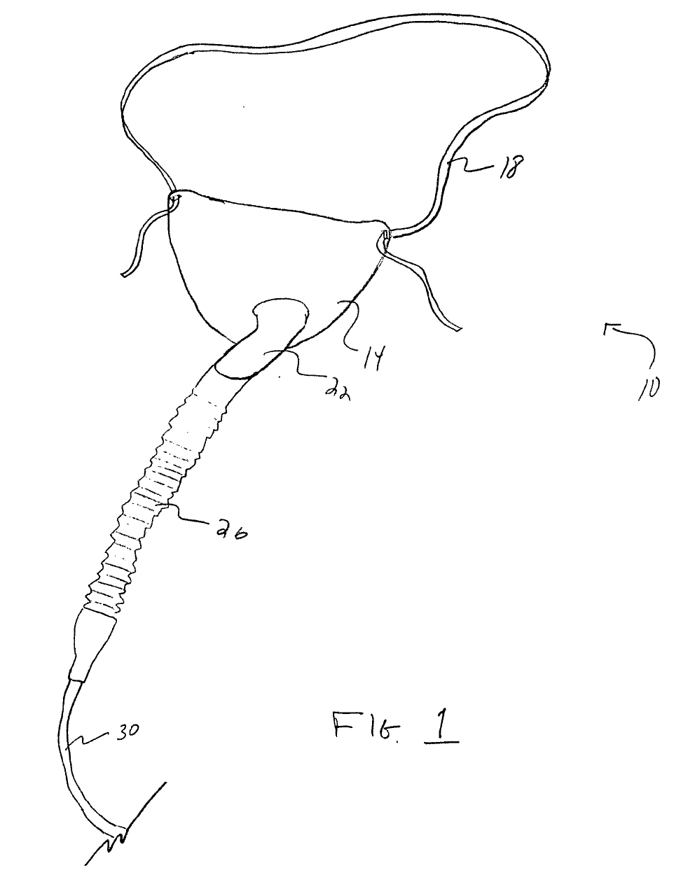

FIG. 1 is a front view of a tracheostomy mask 10. The tracheostomy mask comprises a mask 14 that can fit over a tracheostomy tube typically located in the patient's neck. The mask 10 has an adjustable band 18 to securely hold the mask in place. The mask 10 also has a swivel tubing 22 extending from it; however, the disclosed invention will also work with a mask that has a fixed tubing. The swivel tubing 22 is in communication with an oxygen tube 26. The oxygen tube 26 is in fluid communication with a hose 30; the hose 30 may be attached to a humidifier, oxygen supply, or any other medically necessary device. It should be noted that the disclosed invention will operate if the hose 30 is attached to any device, or if the hose 30 is removed. The oxygen tube 26 is removeably attachable to the tracheostomy mask 10. The oxygen tube 26 may have an accordion type shape as shown in FIG. 1.

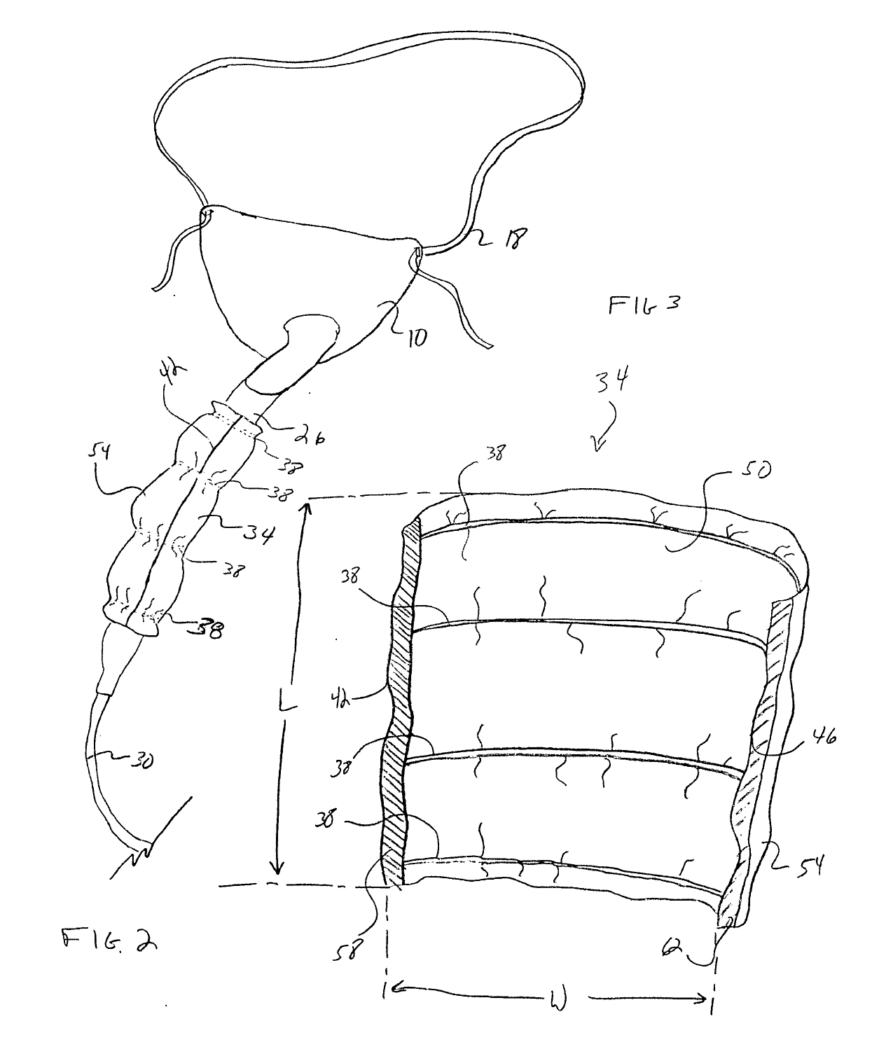

FIG. 2 shows the disclosed oxygen tube cover 34 attached to the oxygen tube 26 in a closed configuration. The oxygen tube cover 34 comprises a soft material such as, but not limited to: felt, velour, flannel, fleece, cotton and wool. The oxygen tube cover has several bands of elastic 38 attached to the oxygen tube cover 34 in order to securely hold the oxygen tube cover 34 to the oxygen tube 26. If the oxygen tube 26 is of the accordion type shape as shown in FIG. 1, then the several bands of elastic 38 will be able to securely hold the oxygen tube cover 34 because the bands of elastic 38 will tend to grab the indented areas of the accordion shape of the oxygen tube 26. The vertical line 42 is the first end 42 of the oxygen tube cover 34. The second end 46 of the oxygen tube cover is shown in FIG. 3 and is not visible in this view. The tactile system for an oxygen tube comprises the oxygen tube cover 34 and the oxygen tube 26.

FIG. 3 shows the disclosed oxygen tube cover 34 in an opened configuration. In this configuration, the first end 42 of the oxygen tube cover 34 is plainly visible as is the second end 46 of the oxygen tube cover 34. The oxygen tube cover 34 has an inner surface 50 that is adjacent to the oxygen tube 26, when the oxygen tube cover 34 is installed on the oxygen tube 26. The oxygen tube cover 34 also has an outer surface 54 that is on an opposite side to the inner surface 50. Near or on the first end 42 of the oxygen tube cover 34, and on the inner surface 50 is a first connecting means 58. Near the or on the second end 46 of the oxygen tube cover 34, on the outer surface is a second connecting means 62. When the oxygen tube cover 34 is attached to an oxygen tube, the oxygen tube cover 34 is wrapped around the tube and the first connecting means 58 is removeably attachable to the second connecting means 62. The first connecting means 58 and second connecting means 62 may comprise any suitable means of connecting, including but not limited to a hook and loop, snap, button and button hole, zipper.

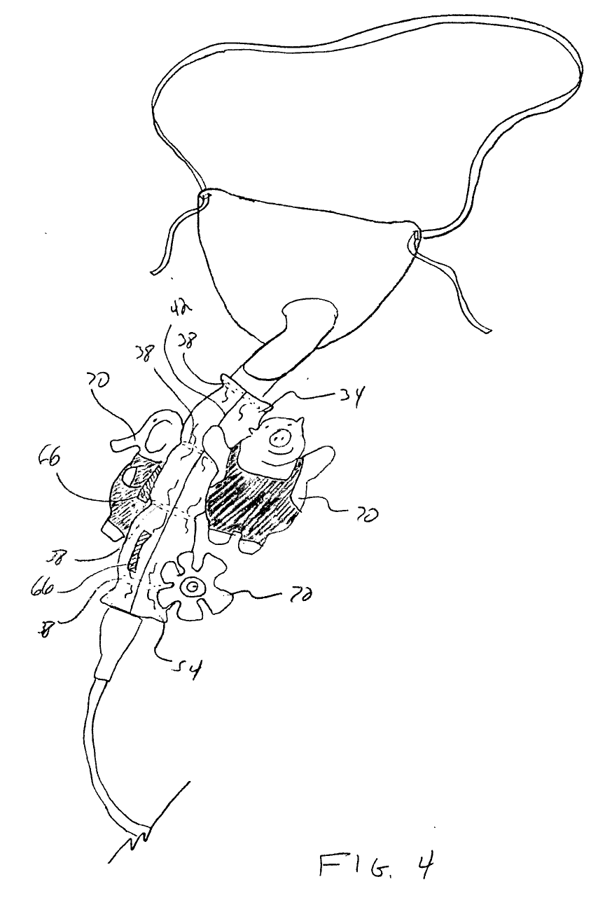

FIG. 4 shows another embodiment of the oxygen tube cover 34. This embodiment is generally the same as the embodiment disclosed in FIGS. 2-3, except for the addition of the ability to removeably attach at least one soft stuffed toy 70 to the outer surface 54 of the oxygen tube cover 34 via a toy connecting means 66 located on the soft stuffed toy 70 and the outer surface 54 of the oxygen tube cover 34. The toy connecting means 66 may comprise any suitable means of connecting, including but not limited to a hook and loop, snap, button and button hole, zipper.

The oxygen tube cover 34 will have a length “L”. L will have a value so that the oxygen tube cover 34 will generally cover the entire oxygen tube 26. However, L may be less or greater than a length required to completely cover the entire oxygen tube 26. The oxygen tube cover 34 will have a width “W”. W will have a value so that the oxygen tube cover 34 will generally wrap once around the oxygen tube 26, with the a first connecting means 58 attachable to the second connecting means 62. W may be defined as the distance from the first end 42 to the second end 46 along the oxygen tube cover 34.

The tactile apparatus and system for oxygen tube has many advantages. The disclosed invention allows a young patient to touch and play with the oxygen tube cover. The invention will discourage parents from telling their child to stop touching and/or playing with the oxygen tube that is attached to the tracheostomy tube. The invention will help the young patient to explore his or her surroundings by touch, sight, and smell. The invention will help prevent the young patient from being introverted, inactive, disengaged due to being told too often to “don't touch that” and/or “don't play with that”.

It should be noted that the terms “first”, “second”, and “third”, and the like may be used herein to modify elements performing similar and/or analogous functions. These modifiers do not imply a spatial, sequential, or hierarchical order to the modified elements unless specifically stated.

While the disclosure has been described with reference to several embodiments, it will be understood by those skilled in the art that various changes may be made and equivalents may be substituted for elements thereof without departing from the scope of the disclosure. In addition, many modifications may be made to adapt a particular situation or material to the teachings of the disclosure without departing from the essential scope thereof. Therefore, it is intended that the disclosure not be limited to the particular embodiments disclosed as the best mode contemplated for carrying out this disclosure, but that the disclosure will include all embodiments falling within the scope of the appended claims.

What is claimed is:

1. A tactile system for an oxygen tube attachable to a tracheostomy mask, the system comprising:
an oxygen tube;
an oxygen tube cover removeably attached to the oxygen tube, the oxygen tube cover comprising:
a first end;
a second end located on an end opposite the first end;
a width defined generally by the distance along the oxygen tube cover from the first end to the second end;
a length;
an inner surface;
an outer surface located on a surface opposite the inner surface;
a plurality of elastic bands attached to the oxygen tube cover generally parallel to the width;
a first connecting means located on the inner surface and generally at the first end;
a second connecting means located on the outer surface and generally at the second end; and
wherein the oxygen tube cover is generally made of a soft material.

2. The tactile system of claim 1, wherein the soft material is selected from the group consisting of felt, velour, flannel, fleece, cotton and wool.

3. The tactile system of claim 1, wherein the oxygen tube is configured to be removeably attachable to a tracheostomy mask.

4. The tactile system of claim 1, wherein the first connecting means and the second connecting means are selected from the group consisting of hook and loop, snap, button and button hole, zipper.

5. The tactile system of claim 1, wherein the length is generally equally to the length of the oxygen tube.

6. The tactile system of claim 1, wherein the width is configured so that the oxygen tube cover can wrap generally once around the oxygen tube allowing the first connecting means to removeably attach to the second connecting means.

7. The tactile system of claim 1, wherein the width is slightly greater than the circumference of the oxygen tube such that the oxygen tube cover can wrap generally once around the oxygen tube allowing the first connecting means to removeably attach to the second connecting means.

8. The tactile system of claim 1, wherein the oxygen tube cover further comprises:
at least one toy connecting means located on the outer surface;
at least one stuffed toy with a toy connecting means located on it;
wherein the at least one stuffed toy is removeably attachable to the outer surface via the toy connecting means.

9. The tactile system of claim 8, wherein the toy connecting means is selected from the group consisting of hook and loop, snap, button and button hole, zipper.

10. The tactile system of claim 8, wherein the stuffed toy is selected from the group consisting of a stuffed animal, stuffed movie character, stuffed television character, stuffed cartoon character, stuffed automobile and stuffed flower.

11. A tactile apparatus for an oxygen tube attachable to a tracheostomy mask, the tactile apparatus comprising:
an oxygen tube cover removeably attachable to an oxygen tube, the oxygen tube cover comprising:
a first end;
a second end located on an end opposite the first end;
a width defined generally by the distance along the oxygen tube cover from the first end to the second end;
a length;
an inner surface;
an outer surface located on a surface opposite the inner surface;
a plurality of elastic bands attached to the oxygen tube cover generally parallel to the width;
a first connecting means located on the inner surface and generally at the first end;
a second connecting means located on the outer surface and generally at the second end; and
wherein the oxygen tube cover is generally made of a soft material.

12. The tactile apparatus of claim 11, wherein the soft material is selected from the group consisting of felt, velour, flannel, fleece, cotton and wool.

13. The tactile apparatus of claim 11, wherein the oxygen tube is configured to be removeably attachable to a tracheostomy mask.

14. The tactile apparatus of claim 11, wherein the first connecting means and the second connecting means are selected from the group consisting of hook and loop, snap, button and button hole, zipper.

15. The tactile apparatus of claim 11, wherein the length is generally equally to the length of the oxygen tube.

16. The tactile apparatus of claim 11, wherein the width is configured so that the oxygen tube cover can wrap generally once around the oxygen tube allowing the first connecting means to removeably attach to the second connecting means.

17. The tactile apparatus of claim 11, wherein the width is slightly greater than the circumference of the oxygen tube such that the oxygen tube cover can wrap generally once around the oxygen tube allowing the first connecting means to removeably attach to the second connecting means.

18. The tactile apparatus of claim 11, wherein the oxygen tube cover further comprises:
at least one toy connecting means located on the outer surface;
at least one stuffed toy with a toy connecting means located on it;
wherein the at least one stuffed toy is removeably attachable to the outer surface via the toy connecting means.

19. The tactile apparatus of claim 18, wherein the toy connecting means is selected from the group consisting of hook and loop, snap, button and button hole, zipper.

20. The tactile apparatus of claim 18, wherein the stuffed toy is selected from the group consisting of a stuffed animal, stuffed movie character, stuffed television character, stuffed cartoon character, stuffed automobile and stuffed flower.
